(19) United States
(12) Patent Application Publication
(10) Pub. No.: US 2024/0335359 A1
(43) Pub. Date: Oct. 10, 2024

(54) Ostomy Re-Feeder Apparatus

(71) Applicant: Joy Cameron-Lawson, Hialeah, FL (US)

(72) Inventor: Joy Cameron-Lawson, Hialeah, FL (US)

(21) Appl. No.: 18/357,456

(22) Filed: Jul. 24, 2023

Related U.S. Application Data
(60) Provisional application No. 63/456,118, filed on Mar. 31, 2023.

(51) Int. Cl. A61J 15/00

(52) U.S. Cl. CPC A61J 15/0015; A61J 15/0057; A61J 15/0061; A61J 15/0096

(57) ABSTRACT

An ostomy re-feeder device is provided. The device is primarily comprised of at least one re-feeding tube, at least one face plate, and at least one bag. The device is designed to be used for direct stomal feeding and also as an independent ostomy collection system. The face plate attaches around a stoma on a user's body, wherein the re-feeding tube enters the face plate via an opening such that the tube can be placed into the stoma for re-feeding. When used as an ostomy collection system, the bag can be attached to the face plate over the stoma and the cutaway entry located on the silicone flange of the face plate or on the bag itself if accessed via accessory is maintained capped.

### CROSS-REFERENCE TO RELATED APPLICATION

The present application claims priority to, and the benefit of, U.S. Provisional Application No. 63/456,118, which was filed on Mar. 31, 2023, and is incorporated herein by reference in its entirety.

### FIELD OF THE INVENTION

The present invention relates generally to the field of re-feeding devices. More specifically, the present invention relates to an ostomy re-feeder device that can be used to re-feed an individual via a stoma. The device can also be used as an ostomy collection system. Accordingly, the present disclosure makes specific reference thereto. Nonetheless, it is to be appreciated that aspects of the present invention are also equally applicable to other like applications, devices, and methods of manufacture.

### BACKGROUND

Individuals who suffer from intestinal pathologies such as necrotizing enterocolitis, intestinal neoplasm, intestinal trauma, etc., may have to undergo an enterostomy, which results in the small intestine being diverted to an artificial opening in the abdominal wall. Said individuals are at risk for fistulation and total GI failure if the functionality of the remaining intestine is not preserved. In addition, said individuals are at increased risk for electrolyte imbalances, severe dehydration, malnutrition, and sepsis. Enterostomal refeeding is often used as a treatment modality for said individuals. However, no hygienic refeeding options exist for said individuals.

Therefore, there exists a long-felt need in the art for a re-feeding device for individuals who have undergone an enterostomy. More specifically, there exists a long-felt need in the art for an ostomy re-feeder device that can be used for direct stomal feeding. In addition, there also exists a long-felt need in the art for an ostomy re-feeder device that can be used as an ostomy collection system, in addition to being used for direct stomal feeding.

The subject matter disclosed and claimed herein, in one embodiment thereof, comprises an ostomy re-feeder device. The device is primarily comprised of at least one re-feeding tube, at least one face plate, and at least one bag. The device is designed to be used for direct stomal feeding but can also be used as an independent ostomy collection system. The face plate attaches around a stoma on a user's body and the collection bag for effluent collection is attached. The re-feeding tube enters the face plate via an opening on the flange of the face plate such that the tube can be placed into the stoma for re-feeding. The clinician will collect effluent at the distal end of the collection bag via a spigot and refeed it via the proximal end of the feeding tube. In an embodiment wherein the device is used as a standard ostomy collection system, the re-feeder tube is not inserted, the bag is attached to the face plate over the stoma and the cutaway entry located on the flange of the faceplate is maintained capped. Or, for prolapsed cases where the port of entry is on the bag itself this is not accessed or is capped if accessed earlier.

In this manner, the ostomy re-feeder device of the present invention accomplishes all the foregoing objectives and provides a re-feeding device for individuals who have undergone an enterostomy. More specifically, the device can be used for direct stomal feeding. In addition, the device can also be used as an ostomy collection system.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an ostomy re-feeder device. The device is primarily comprised of at least one re-feeding tube, at least one face plate, and at least one bag. The device is designed to be used for direct stomal feeding but can also be used as an independent ostomy collection system.

The face plate of the device attaches to the skin of a user during use, wherein at least one opening of a bottom surface of the face plate is positioned around a stoma area. The face plate is designed within a hydrocolloid surface which allows direct attachment to the user's skin around the stoma. The face plate is preferably comprised of a side wall with at least one opening that allows the first (distal) end of at least one re-feeding tube to be inserted through the side wall and into the stoma within the side wall. In one embodiment, the opening can be covered with at least one cap when not in use. To further support/position the tube within the face plate, the interior space of the face plate may be equipped with a removable accessory or at least one support member that supports the weight and/or position of the tube. In this manner, a user can more precisely position the tube within the face plate via the member, wherein the tube cannot shift position easily.

The device is also comprised of at least one bag that functions as a body-worn, effluent collection receptacle. The bag may have any features commonly found on ostomy bags known in the art. The bag is comprised of at least one gas vent that allows a user to release gas from the bag as needed. In addition, the bag is comprised of at least one spigot at its's distal end that can be used for the release or the collection of effluent from the bag. The spigot should be kept closed to prevent leakage from the bag when not in use.

In one embodiment, the exterior of the bag is comprised at the proximal end of at least one fastener. The fastener is a water-tight, sealed fastener such as, but not limited to, a clip, snap fastener, a rotating fastener, etc., (or other fastener of the like) that allows the bag to attach to the side wall of the face plate. In this manner, the bag is supported by the face plate and also functions as a top surface for the face plate to protect the stoma.

The re-feeding tube may be similar to any type of medical-grade tubing known in the art but has a few different features as it relates to feeding tubes. The tube may be comprised of at least one inflatable member that can be used to prevent leaks after insertion; and also, a well veiled flexible, conforming wire embedded deep within its' fabrication of the feeding tube to prevent expulsion of tube by intra-abdominal pressures. The re-feeder tube may be secured to a user's skin/body via at least one tube anchor such as medical-grade tape, clip for aesthetics and comfort when in or not in use.

The present invention is also comprised of a method of using the device for feeding. First, a device is provided comprised of at least one tube, at least one face plate, and at least one bag. Then, the stoma can be cleaned of all output debris in accordance with known medical cleaning procedures. Then, the face plate can be applied around a stoma onto a user's skin. In one method, the re-feeder tube can be inserted through an opening inside the face plate and threaded directly into the stoma. The re-feeder tube has a very thin, flexible, conforming wire embedded deep within its' fabrication design that offsets expulsion from the stoma due to intra-abdominal/intra-luminal forces. Additionally, the first (distal) end of the re-feeder tube can be inserted through the opening on the flange of the face plate and into the stoma at a desired depth. Next, the inflatable member may be inflated to prevent leakage around the tube and/or to provide anchoring of the tube to prevent expulsion due to abdominal pressure. After insertion, the re-feeder tube can be secured to the skin/clothing of the user. Next, the collection bag can be secured to the face plate via the fastener of the bag.

The present invention is also comprised of a method of using the device for effluent collection. First, a device is provided comprised of at least one tube, at least one face plate, and at least one bag. Then, the stoma can be cleaned of all output debris in accordance with known medical cleaning procedures. Then, the face plate can be applied around a stoma onto a user's skin. Then, the bag can be secured to the face plate via the fastener of the bag. Next, the first (distal) end of the tube can be inserted through the opening of the side wall and into the stoma at a desired depth. Then, the inflatable member may be inflated to prevent leakage around the tube and/or to provide anchoring of the tube to prevent expulsion due to abdominal pressure. Then, the tube can be secured to the skin/clothing of the user via the anchor and can be used as needed. Next, the proximal (second) end of the tube is used for refeeding. A spigot is on the distal end of the collection bag is used to empty/collect effluent from the bag. The spigot must be kept closed at all times after use.

Accordingly, the ostomy re-feeder device of the present invention is particularly advantageous as it provides a re-feeding device for individuals who have undergone an enterostomy. More specifically, the device can be used for direct stomal feeding. In addition, the device can also be used as an ostomy collection system. In this manner, the ostomy re-feeder device overcomes the limitations of existing re-feeding devices known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a face plate of an ostomy re-feeder device of the present invention attached around a user's stoma in accordance with the disclosed architecture;

FIG. 2 illustrates a perspective view of one potential embodiment of a support member and a face plate of an ostomy re-feeder device of the present invention in accordance with the disclosed architecture;

FIG. 3 illustrates a perspective view of a tube of one potential embodiment of an ostomy re-feeder device of the present invention in accordance with the disclosed architecture;

FIG. 4 illustrates a perspective view of a bag of one potential embodiment of an ostomy re-feeder device of the present invention in accordance with the disclosed architecture;

FIG. 5 illustrates a flowchart of a method of using one potential embodiment of an ostomy re-feeder device of the present invention for re-feeding in accordance with the disclosed architecture;

FIG. 6 illustrates a flowchart of a method of using one potential embodiment of an ostomy re-feeder device of the present invention for re-feeding in accordance with the disclosed architecture;

FIG. 7 illustrates a flowchart of a method of using one potential embodiment of an ostomy re-feeder device of the present invention for re-feeding in accordance with the disclosed architecture; and

FIG. 8 illustrates a flowchart of a method of using one potential embodiment of an ostomy re-feeder device of the present invention for effluent collection in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a re-feeding device for some individuals who have undergone an enterostomy. More specifically, there exists a long-felt need in the art for an ostomy re-feeder device that can be used for direct stomal feeding. In addition, there also exists a long-felt need in the art for an ostomy re-feeder device that can be used as an ostomy collection system, in addition to being used for direct stomal feeding.

The present invention, in one exemplary embodiment, is comprised of an ostomy re-feeder device primarily comprised of at least one re-feeding tube, at least one face plate, and at least one bag. The device is designed to be used for direct stomal feeding. In addition, the device can be used as an independent ostomy collection system.

The face plate of the device attaches to the skin of a user during use, wherein at least one opening of a bottom surface of the face plate is positioned around a stoma area. To allow the face plate to attach to a user's skin, the bottom surface of the face plate may be comprised of at least one fastener. The face plate is preferably comprised of a side wall and has at least one opening. The opening allows the first (distal) end of at least one re-feeding tube to be inserted through the side wall and into the stoma. In one embodiment, the opening can be covered with at least one cap when not in use. To further support/position the tube within the face plate, the interior space of the face plate may be adapted to receive at least one support member that supports the weight and/or position of the tube. In this manner, a user can more precisely position the tube within the face plate via the member, and wherein the tube cannot shift position easily.

The device is also comprised of at least one bag. The bag functions as a body-worn, effluent collection receptacle. The bag may have any features commonly found on ostomy bags known in the art. The bag is comprised of at least one gas vent that allows a user to release gas from the bag as needed. In addition, the bag is comprised of at least one spigot that can be used for the release of effluent from the bag. In another embodiment, the spigot can be used to collect effluent from the bag. The spigot must be kept closed to prevent leakage from the bag.

In one embodiment, the exterior of the bag is comprised of at least one fastener that is a water-tight, sealed fastener such as, but not limited to, a clip, snap fastener, a rotating fastener, etc., (or other fastener of the like) that allows the bag to attach to the side wall of the face plate. In this manner, the bag is supported by the face plate and also functions as a top surface for the face plate to protect the stoma.

The re-feeding tube may be similar to any type of medical-grade tubing known in the art but has a few different features as it relates to feeding tubes. The tube may be comprised of at least one inflatable member that can be used to prevent leaks after insertion. Additionally, the re-feeder tube consists of a thin, flexible conforming wire embedded deep within its' fabrication design that overrides the expelling intestinal forces. The tube may further be secured to a user's skin/body via at least one tube anchor, such as medical-grade tape, clip, etc.

The present invention is also comprised of a method of using the device for feeding. First, a device is provided comprised of at least one tube, at least one face plate, and at least one bag. Then, the stoma can be cleaned of all output debris in accordance with known medical cleaning procedures. Then, the face plate can be applied around a stoma onto a user's skin. In one method, the support member can then be placed inside the face plate and used to support/position the tube within the stoma. Next, the bag can be secured to the face plate via the fastener of the bag. Then, the first (distal) end of the tube can be inserted through the opening of the side wall and into the stoma at a desired depth. Next, the inflatable member may be inflated to prevent leakage around the tube and/or to provide anchoring of the tube to prevent expulsion due to abdominal pressure. Then, the tube can be secured to the skin/clothing of the user via the anchor and can be used as needed for feeding via the tube.

The present invention is also comprised of a method of using the device for effluent collection. First, a device is provided comprised of at least one tube, at least one face plate, and at least one bag. Then, the stoma can be cleaned of all output debris in accordance with known medical cleaning procedures. Then, the face plate can be applied around a stoma onto a user's skin. Then, the bag can be secured to the face plate via the fastener of the bag. Next, the first (distal) end of the tube can be inserted through the opening of the side wall and into the stoma at a desired depth. Then, the inflatable member may be inflated to prevent leakage around the tube and/or to provide anchoring of the tube to prevent expulsion due to abdominal pressure. Then, the tube can be secured to the skin/clothing of the user via the anchor and can be used as needed for feeding.

Accordingly, the ostomy re-feeder device of the present invention is particularly advantageous as it provides a re-feeding device for some individuals who have undergone an enterostomy. More specifically, the device can be used for direct stomal feeding. In addition, the device can also be used as an ostomy collection system. In this manner, the ostomy re-feeder device overcomes the limitations of existing re-feeding devices known in the art.

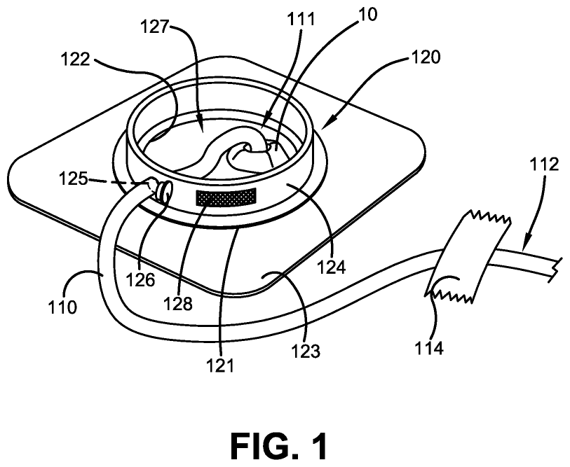

Referring initially to the drawings, FIG. 1 illustrates a perspective view of one potential embodiment of a face plate 120 of an ostomy re-feeder device 100 of the present invention attached around a user's stoma in accordance with the disclosed architecture. The device 100 is primarily comprised of at least one re-feeding tube, at least one face plate 120, and at least one bag 140. The device 100 is designed to be used for direct stomal feeding. In addition, the device 100 can also be used as an independent ostomy collection system.

The face plate 120 of the device 100 attaches to the skin of a user during use, wherein at least one opening 122 of a bottom surface 121 of the face plate 120 is positioned around a stoma 10 area. The face plate 120 is preferably made from a firm silicone material embedded on a moldable hydrocolloid base. To decrease skin irritation at the stoma site the hydrocolloidal base of the face plate 120 may be impregnated with at least one neutralizing agent 128 known in the art to prevent irritation from stoma leaks onto the user's skin.

To allow the face plate 120 to attach to a user's skin, the bottom surface 121 of the face plate 121 may be comprised of at least one fastener 123. The fastener 123 is preferably a skin-safe adhesive such as, but not limited to, hydrocolloid. The fastener 123 secures the face plate 120 in place during use.

The face plate 120 is preferably comprised of a side wall 124. The side wall 124 is preferably circular but may be any shape. In one embodiment, the side wall 124 is comprised of at least one opening 125. The opening 125 allows a first (distal) end 111 of at least one re-feeding tube 110 to be inserted through the side wall 124 and into the stoma 110 within the side wall 124. In one embodiment, the opening 125 can be covered with at least one cap 126 when not in use. The cap 126 may also be comprised of a gasket cap that further seals the opening 125 via the gasket structure to prevent leakage from the stoma 10 from exiting the face plate 120 when not in use.

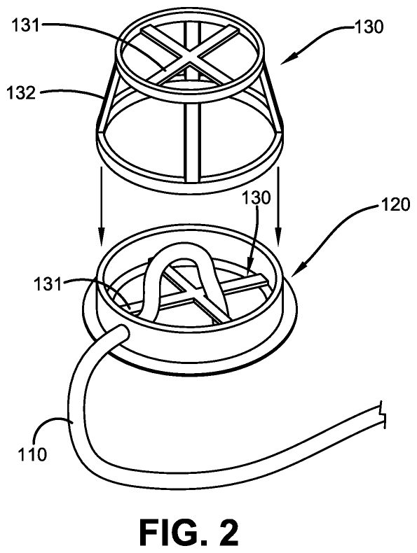

To further support/position the tube 110 within the face plate 120, the interior space 127 of the face plate 120 may be adapted to receive at least one support member 130, as seen in FIG. 2. The support member 130 is preferably circular but may be any shape. The member 130 has a plurality of vertical frame members 131 and horizontal frame members 132 that support the weight and/or position of the tube 110. In this manner, a user can more precisely position the tube 110 within the face plate 110 via the member 130, and wherein the tube 110 cannot shift position easily.

The device 100 is also comprised of at least one bag 140. The bag 140 functions as a body-worn, effluent collection receptacle. The bag 140 may have any features commonly found on ostomy bags known in the art. The bag 140 is preferably made from a polyurethane plastic but may be made from any medical-grade material. The bag 140 is comprised of at least one gas vent 141 that allows a user to release gas from the bag 140 as needed. In addition, the bag 140 is comprised of at least one spigot 145. The spigot 145 can be used for the release of effluent from the bag 140. In another embodiment, the spigot 145 can be used to collect effluent from the bag 140. The spigot 145 should be closed when not in use to prevent leakage from the bag 140.

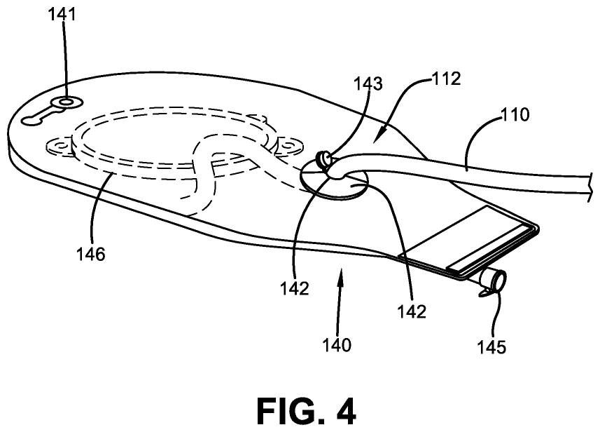

In one embodiment, the bag 140 is comprised of at least one feeding tube ring 142. This embodiment of the device 100 is used when the stoma 10 is prolapsed beyond the side wall 124. The ring 142 can be punctured by the tube 110 to allow the tube 110 to pass through the bag 140 (as seen in FIG. 4), wherein the bag 140 still covers the stoma 10. In one embodiment, the ring 142 is an adhesive ring that can be attached anywhere on the bag 140. A user can then puncture the bag 140 with the tube 110 to allow the tube 110 to pass through the bag 140 and into the face plate 120, as noted. The ring 142 may be comprised of at least one cap 143 that seals the opening 147 formed by the tube 110 when the tube 110 is not in use (i.e., when the tube 110 is connected to the spigot 145 and used to fill the bag 140).

In one embodiment, the exterior of the bag 140 is comprised of at least one fastener 146. The fastener 146 is a water-tight, sealed fastener such as, but not limited to, a clip, snap fastener, a rotating fastener, etc. (or other fastener of the like) that allows the bag 145 to attach to the side wall 124 of the face plate 120. In this manner, the bag 140 is supported by the face plate 120 and also functions as a top surface for the face plate 120 to protect the stoma 10. In one embodiment, the fastener 146 and side wall 124 are each comprised of at least one alignment marking 150 to allow the face plate 110 and fastener 146 to be properly aligned for secure attachment.

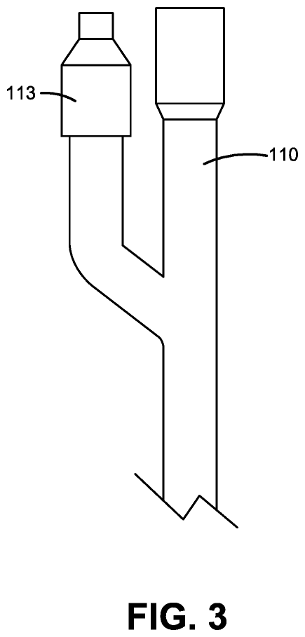

The re-feeding tube 110 The re-feeding tube may be similar to any type of medical-grade tubing known in the art but has a few different features as it relates to feeding tubes. In one embodiment, the tube is comprised of a flexible, conforming, micro-wire embedded deep within the fabrication of the tube. The tube 110 may also be comprised of at least one inflatable member 113 that can be used to prevent leaks after insertion, as seen in FIG. 3. The tube 110 may further be secured to a user's skin/body via at least one tube anchor 114, such as medical-grade tape, clip, etc. The anchor 114 can be adjusted along the tube 110 to allow appropriate anchor approximation for the indwelling depth at the first (distal) end 111 of the tube 110 while allowing ample tube length proximally for patient comfort and hygienic practices.

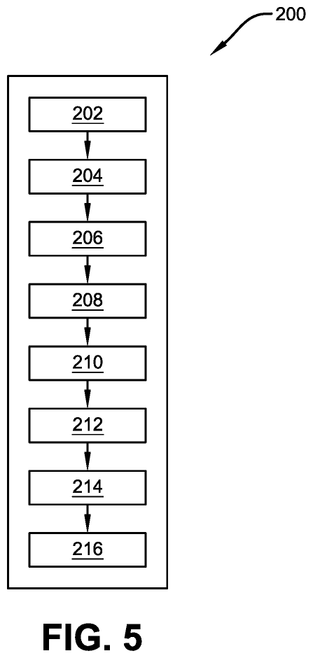

The present invention is also comprised of a method of using 200 the device 100 for feeding, as seen in FIG. 5. First, a device 100 is provided comprised of at least one tube 110, at least one face plate 120, and at least one bag 140 [Step 202]. Then, the stoma 10 can be cleaned of all output debris in accordance with known medical cleaning procedures [Step 204]. Then, the face plate 120 can be applied around a stoma 10 onto a user's skin [Step 206]. Next, the bag 140 can be secured to the face plate 120 via the fastener 146 of the bag 140 [Step 208]. Then, the first (distal) end 111 of the tube 110 can be inserted through the opening 125 of the side wall 124 and into the stoma 10 at a desired depth [Step 210]. Next, the inflatable member 113 may be inflated to prevent leakage around the tube 110 and/or to provide anchoring of the tube 110 to prevent expulsion due to abdominal pressure [Step 212]. Then, the tube 110 can be secured to the skin/clothing of the user via the anchor 114 and can be used as needed for feeding via the tube [Step 214].

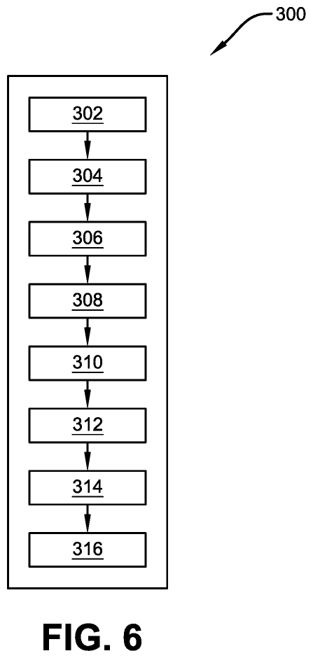

The present invention is also comprised of a method of using 300 the device 300 for feeding, as seen in FIG. 6. First, a device 100 is provided comprised of at least one tube, at least one face plate 120, at least one support member 130, and at least one bag 140 [Step 302]. Then, the stoma 10 can be cleaned of all output debris in accordance with known medical cleaning procedures [Step 304]. Then, the face plate 120 can be applied around a stoma 10 onto a user's skin [Step 306]. Next, the support member 130 can be placed inside the face plate 120 [Step 308]. Then, the bag 140 can be secured to the face plate 120 via the fastener 146 of the bag 140 [Step 310]. Next, the first (distal) end 111 of the tube 110 can be inserted through the opening 125 of the side wall 124 and into the stoma 10 at a desired depth, wherein the tube 110 can be positioned/supported using the support member 130 [Step 312]. Then, the inflatable member 113 may be inflated to prevent leakage around the tube 110 and/or to provide added anchoring of the tube 110 to prevent expulsion due to abdominal pressure [Step 314]. Then, the tube 110 can be secured to the skin/clothing of the user via the anchor 114 and can be used as needed for feeding via the tube [Step 316].

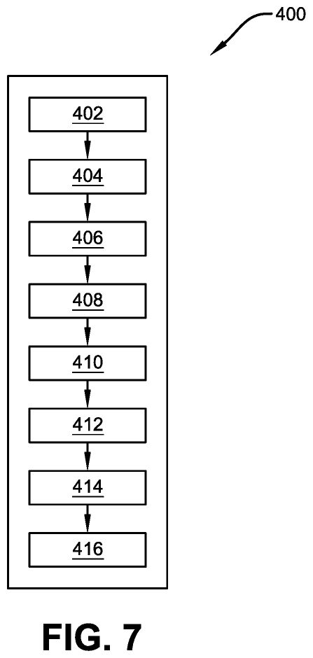

The present invention is also comprised of a method of using 400 the device 100 for feeding, as seen in FIG. 7. First, a device 100 is provided comprised of at least one tube, at least one face plate 120, and at least one bag 140 [Step 402]. Then, the stoma 10 can be cleaned of all output debris in accordance with known medical cleaning procedures [Step 404]. Then, the face plate 120 can be applied around a stoma 10 onto a user's skin [Step 406]. Then, the bag 140 can be secured to the face plate 120 via the fastener 146 of the bag 140 [Step 408]. Next, the first (distal) end 111 of the tube 110 can be inserted through the opening 125 of the side wall 124 and into the stoma 10 at a desired depth [Step 410]. If the stoma 10 is prolapsed beyond the side walls 124, the feeding tube ring 142 can be applied to the bag 140 at the area closest to the stoma 10 such that the tube 110 can be inserted through an opening 147 of the ring 142 and through the bag 140 [Step 412]. Then, the inflatable member 113 may be inflated to prevent leakage around the tube 110 and/or to provide anchoring of the tube 110 to prevent expulsion due to abdominal pressure [Step 414]. Then, the tube 110 can be secured to the skin/clothing of the user via the anchor 114 and can be used as needed for feeding via the tube [Step 416].

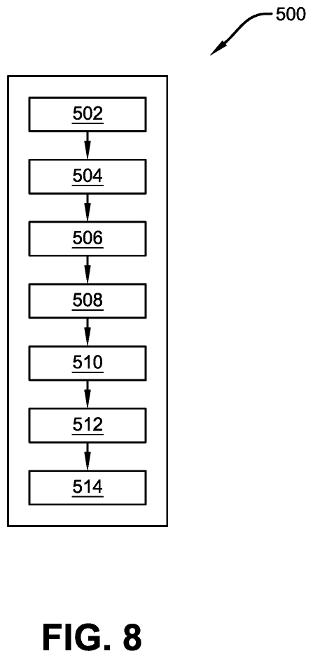

The present invention is also comprised of a method of using 500 the device 100 for effluent collection, as seen in FIG. 8. First, a device 100 is provided comprised of at least one tube, at least one face plate 120, and at least one bag 140 [Step 502]. Then, the stoma 10 can be cleaned of all output debris in accordance with known medical cleaning procedures [Step 504]. Then, the face plate 120 can be applied around a stoma 10 onto a user's skin [Step 506]. Then, the bag 140 can be secured to the face plate 120 via the fastener 146 of the bag 140 [Step 508]. Next, the first (distal) end 111 of the tube 110 can be inserted through the opening 125 of the side wall 124 and into the stoma 10 at a desired depth [Step 510]. Next, the second (proximal) end 112 of the tube 110 can be connected to a feeding extension tubing to a feeding pump for refeeding [Step 512]. Then, the inflatable member 113 may be inflated to prevent leakage around the tube 110 and/or to provide anchoring of the tube 110 to prevent expulsion due to abdominal pressure [Step 514]. Then, the tube 110 can be secured to the skin/clothing of the user via the anchor 114 as needed for refeeding via the tube [Step 516].

It should be noted that any step of any method 200,300,400,500 may be combined into a method of using the device 100.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “ostomy re-feeder apparatus” and “device” are interchangeable and refer to the ostomy re-feeder device 100 of the present invention.

Notwithstanding the foregoing, the ostomy re-feeder device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the ostomy re-feeder device 100 as shown in the FIGS. are for illustrative purposes only, and that many other sizes and shapes of the ostomy re-feeder device 100 are well within the scope of the present disclosure. Although the dimensions of the ostomy re-feeder device 100 are important design parameters for user convenience, the ostomy re-feeder device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An ostomy re-feeder device comprising:
a re-feeding tube comprised of an inflatable member;
a face plate comprised of a bottom surface comprised of a first opening and a first fastener, and side wall comprised of a second opening and a cap; and
a bag comprised of a second fastener, spigot, and a gas vent.

2. The ostomy re-feeder device of claim 1, wherein the re-feeding tube is comprised of a micro-wire embedded tubing.

3. The ostomy re-feeder device of claim 1, wherein the base is impregnated with a neutralizing agent.

4. The ostomy re-feeder device of claim 1, wherein the first fastener is comprised of a hydrocolloid fastener.

5. The ostomy re-feeder device of claim 1, wherein the face plate is comprised of a silicone material.

6. The ostomy re-feeder device of claim 1, wherein the cap is comprised of a gasket cap.

7. The ostomy re-feeder device of claim 1, wherein the bag is comprised of a polyurethane material.

8. An ostomy re-feeder device comprising:
a re-feeding tube comprised of an inflatable member;
a face plate comprised of a bottom surface comprised of a first opening and a first fastener, and a side wall comprised of a second opening and a first cap;
a support member; and
a bag comprised of a second fastener, a spigot, and a gas vent.

9. The ostomy re-feeder device of claim 8, wherein the support member can be positioned inside the face plate.

10. The ostomy re-feeder device of claim 8 further comprised of an adhesive ring.

11. The ostomy re-feeder device of claim 10, wherein the adhesive ring is comprised of a second cap.

12. The ostomy re-feeder device of claim 8, wherein the second fastener is comprised of a water-tight, sealed fastener.

13. The ostomy re-feeder device of claim 12, wherein the fastener is comprised of a clip, a snap fastener, or a rotating fastener.

14. The ostomy re-feeder device of claim 10, wherein the fastener receives the side wall.

15. The ostomy re-feeder device of claim 8 further comprised of a tube anchor.

16. The ostomy re-feeder device of claim 15, wherein the tube anchor is comprised of a medical-grade tape.

17. The ostomy re-feeder device of claim 15, wherein the tube anchor is comprised of a clip.

18. A method of using an ostomy re-feeder device, the method comprising the following steps:
providing an ostomy re-feeder device comprising a tube, a face plate, and a bag;
cleaning a stoma of a user;
applying the face plate around the stoma;
securing the bag to the face plate;
inserting a first distal end of the tube into a sidewall of the face plate and into the stoma; and
securing the tube to a user's skin or a user's clothing via a tube anchor.

19. The method of claim 18 further comprised of a step of attaching a second proximal end of the re-feeder tube to a feeding extension tubing for bolus or continuous refeeding using a feeding pump.

20. The method of claim 18 further comprised of a step of puncturing the bag with the tube after applying an adhesive ring to the bag.
